(19) United States
(12) Patent Application Publication
(10) Pub. No.: US 2009/0326230 A1
(43) Pub. Date: Dec. 31, 2009

(54) PROCESS FOR PREPARING SOLIFENACIN AND ITS SALTS

(75) Inventors: Vijayavitthal Thippannachar Mathad, Nashik (IN); Jaydeepkumar Dahyabhai Lilakar, Gujrat (IN); Goverdhan Gilla, Hyderabad (IN); Sriramireddy Kikkuru, Guntur (IN); Raveendra Reddy Chinta, Chittoor (IN); Swaroopa Dudipala, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: Dr. Reddy's Laboratories Ltd., Hyderabad, Andhra Pradesh (IN); Dr. Reddy's Laboratories, Inc., Bridgewater, NJ (US)

(21) Appl. No.: 12/374,335

(22) PCT Filed: Jul. 18, 2007
(86) PCT No.: PCT/US07/73782

§ 371 Date: Jan. 19, 2009

Related U.S. Application Data
(60) Provisional application No. 60/807,781, filed on Jul. 19, 2006.
(60) Provisional application No. 60/868,399, filed on Dec. 4, 2006.

(30) Foreign Application Priority Data
Jul. 27, 2006 (IN) 1328/CHE/2006

(51) Int. Cl. C07D 453/02

(52) U.S. Cl. 546/137

(57) ABSTRACT

The present invention relates to solifenacin in solid form and a process for its preparation and to a process for the preparation of (1S)-1-Phenyl-1,2,3,4-tetrahydro-isoquinoline, a key intermediate in the synthesis of solifenacin and its salts.

### FIELD OF THE INVENTION

The present invention relates to a process for the preparation of solifenacin and its pharmaceutically acceptable salts. More specifically, the present invention relates to solifenacin in solid form and a process for its preparation and to a process for the preparation of (1S)-1-Phenyl-1,2,3,4-tetrahydro-isoquinoline, a key intermediate in the synthesis of solifenacin and its salts.

### BACKGROUND OF THE INVENTION

Solifenacin succinate is described chemically as (1S)-(3R)-1-azabicyclo[2.2.2]oct-3-yl 3,4-dihydro-1-phenyl-2(1H)-isoquinolinecarboxylate compound with butanedioic acid (1:1), and is structurally represented by Formula I.

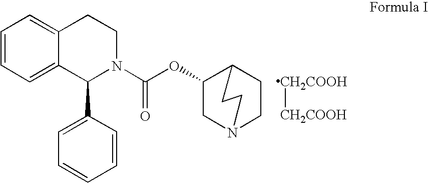

Solifenacin succinate is a muscarinic receptor antagonist useful in the treatment of patients with overactive bladders, urgency and urinary frequency. It is available in the market under the brand name VESIcare® in the form of tablets for oral administration containing 5 or 10 mg of solifenacin succinate.

U.S. Pat. No. 6,017,927 discloses solifenacin and its pharmaceutically acceptable salts, and a process for the preparation of solifenacin and its salts. European Patent No. 1714965 describes compositions containing solifenacin succinate with less impurities and a process for its preparation. European Patent No. 1726304 describes solifenacin or its salts having high purity. Processes for the preparation of solifenacin have also been described in Drugs of the Future 1998, 24(8) 871-874, and Journal of Medicinal Chemistry, 2005, 48, 6597-6606.

The synthesis of solifenacin involves resolution of racemic intermediates, during which there is a greater probability for the isomeric impurities to be carried to the next stages. Thus, in such synthesis, it is desirable to obtain the intermediates in the individual steps in highly purified form for use in the succeeding steps. Crystallinity of intermediates reflects their purity, and is highly desirable since unwanted side reactions involving impurities can be avoided in the subsequent steps of the overall process.

The process of the present invention has advantages of improved yield and increased productivity which affords a significantly greater weight of solifenacin and its pharmaceutically acceptable salts. The process is also industrially scaleable, and cost effective.

### SUMMARY OF THE INVENTION

The present invention relates to a process for the preparation of solifenacin and its pharmaceutically acceptable salts. In particular, the present invention relates to solid solifenacin and a process for its preparation. It also relates to a process for the preparation of (1S)-1-phenyl-1,2,3,4-tetrahydro-isoquinoline, a key intermediate in the synthesis of solifenacin and its salts.

An object of the present invention provides a process for the preparation of solifenacin having Formula VI.

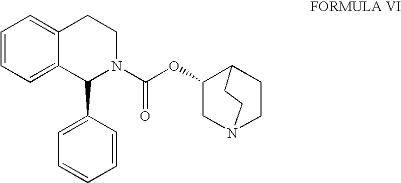

In an aspect, the process comprises of reacting (1S)-alkyl-1-phenyl-1,2,3,4-tetrahydro-2-isoquinolinecarboxylate of Formula IV with less than about 1.5 molar equivalents of (3R)-3-quinuclidinol of Formula V using less than about 1 molar equivalent of a base, to give solifenacin of Formula VI.

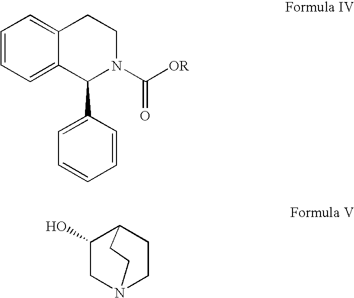

where R is C₁ to C₄ alkyl, aryl, or aralkyl group.

In another aspect, the present invention provides a process for the preparation of (1S)-alkyl-1-phenyl-1,2,3,4-tetrahydro-2-isoquinolinecarboxylate of Formula IV, which process comprises:

a) reacting 1-phenyl-1,2,3,4 tetrahydro-isoquinoline of Formula II with an optically pure tartaric acid in the presence of a suitable organic solvent to afford a tartaric acid salt of (1S) 1-phenyl-1,2,3,4 tetrahydro-isoquinoline of Formula IIa;

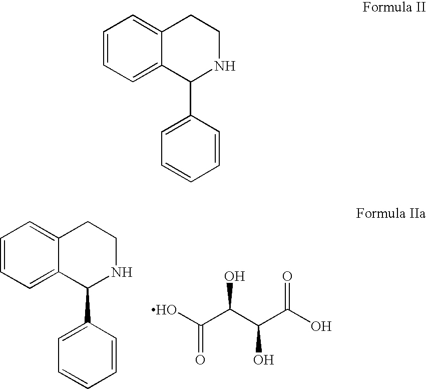

b) reacting the tartaric acid salt of Formula IIa with a suitable base in presence of a suitable solvent to give (1S)-1-phenyl-1,2,3,4 tetrahydro-isoquinoline of Formula III; and

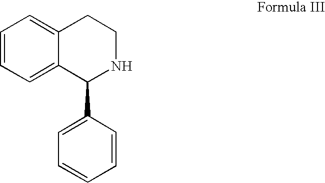

c) reacting (1S) 1-phenyl-1,2,3,4 tetrahydro-isoquinoline of Formula II with alkylchloroformate to give (1S)-alkyl-1-phenyl-1,2,3,4-tetrahydro-2-isoquinolinecarboxylate of Formula IV.

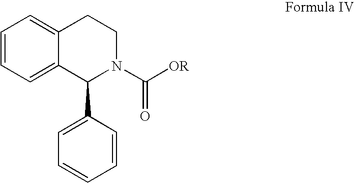

where R is C₁ to C₄ alkyl, aryl, or aralkyl group.

In still another aspect, the present invention provides an inexpensive and commercially viable process for the recovery of 1-phenyl-1,2,3,4 tetrahydro-isoquinoline of Formula II from the mother liquors generated during the resolution stage in the preparation of (1S)-1-phenyl-1,2,3,4 tetrahydro-isoquinoline of Formula III.

In an embodiment, the process comprises treatment of the mother liquor with a suitable base.

One object of the present invention provides solifenacin in a solid form.

In an aspect, the solid is in the form of a crystalline form characterized by an XRPD pattern having significant peaks at about 3.6, 13.4, 14.1, 15.5, 18.6, 19.2, 20.9, 21.7, and 22.9, ±0.2 degrees 2θ.

In another object of the present invention provides substantially pure solifenacin.

Another object of the present invention provides a process for the preparation of pharmaceutically acceptable salts of solifenacin starting from solid solifenacin, or solifenacin prepared according to the process described above.

In another object, the present invention provides a pharmaceutical composition comprising solifenacin or its pharmaceutically acceptable salts prepared according to the process of the present invention, along with one or more pharmaceutically acceptable excipients.

In the process of the present invention control of the number of equivalents of reagents, for example. sodium hydride and quinuclidinol provides advantages of improved yield and isolation of solifenacin free base in a solid form provides increased productivity. This affords a significantly greater weight of solifenacin and its pharmaceutically acceptable salts. The process is also industrially scaleable, and cost effective.

### BRIEF DESCRIPTION OF THE DRAWINGS

FIG. 1 is a schematic representation of a process for the preparation of solifenacin starting from the intermediate compound of Formula II.

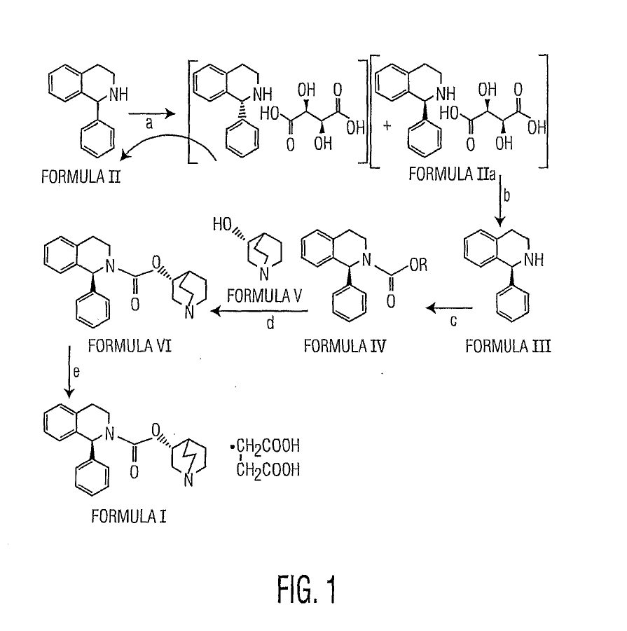

FIG. 2 is an X-ray powder diffraction pattern of crystalline solifenacin prepared in Example 6.

FIG. 3 is an infrared absorption spectrum of crystalline solifenacin prepared in Example 6.

FIG. 4 is a differential scanning calorimetric curve of crystalline solifenacin prepared in Example 6.

FIG. 5 is an X-ray powder diffraction pattern of crystalline solifenacin succinate prepared in Example 9.

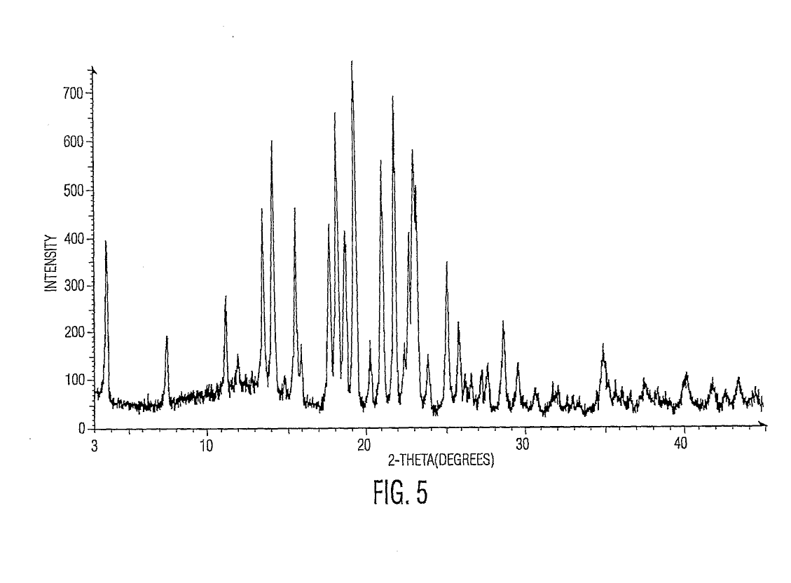

FIG. 6 is an X-ray powder diffraction pattern of crystalline solifenacin hydrochloride prepared in Example 10.

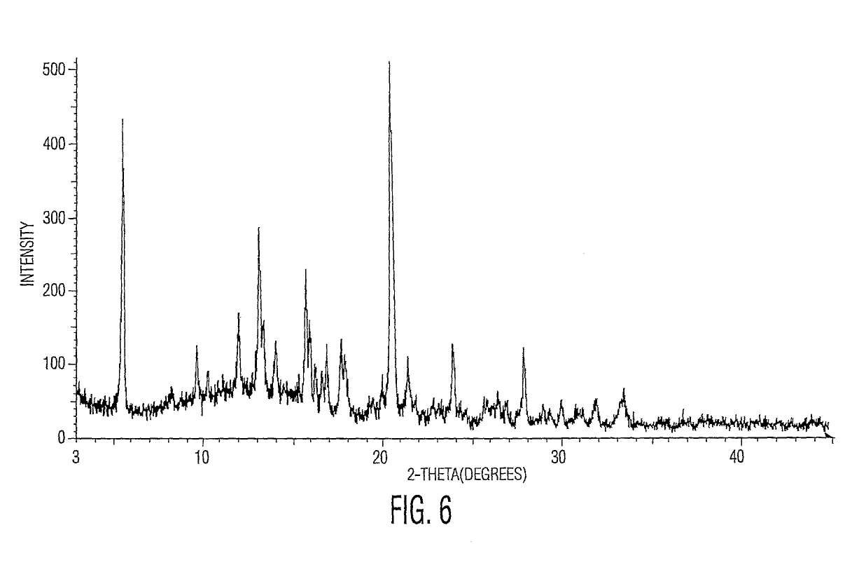

### DETAILED DESCRIPTION OF THE INVENTION

The present invention relates to a process for the preparation of solifenacin and its pharmaceutically acceptable salts. In particular, the present invention relates to solid solifenacin and a process for its preparation. It also relates to a process for the preparation of (1S)-1-phenyl-1,2,3,4-tetrahydro-isoquinoline, a key intermediate in the synthesis of solifenacin and its salts.

One object of the present invention provides solifenacin in a solid form.

Any form of solifenacin such as any crystalline form, amorphous form, hydrated form, or any solvated form is encompassed within the scope of the present invention.

In one embodiment, the solid form of solifenacin is a crystalline form characterized by its X-ray powder diffraction (“XRPD”) pattern, differential scanning calorimetry (“DSC”) curve, and infrared (“IR”) absorption spectrum.

All XRPD data reported herein were obtained using Cu Kα radiation, having the wavelength 1.541 Å and were obtained using a Bruker AXS D8 Advance Powder X-ray Diffractometer.

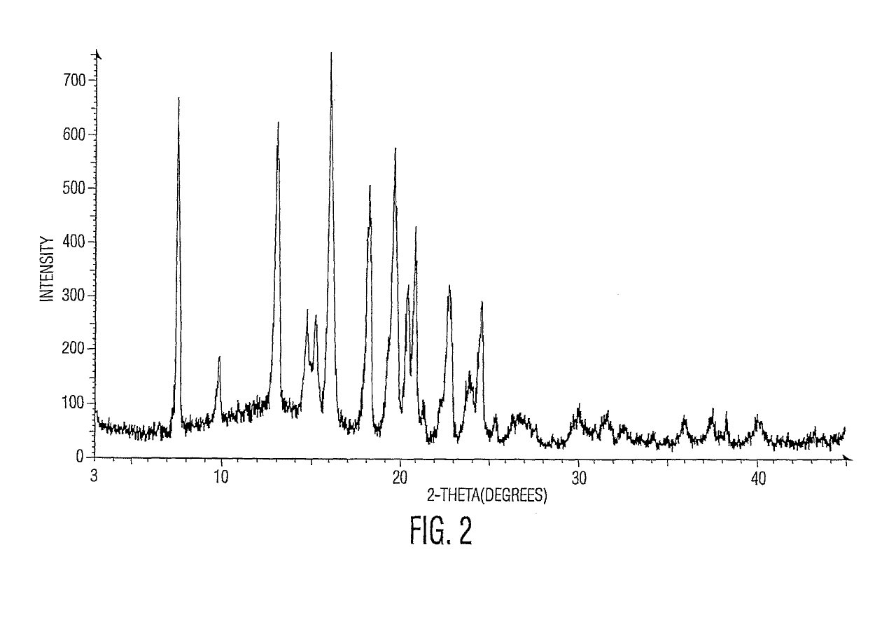

The crystalline form of solifenacin will be designated hereinafter as “crystalline Form A.” It is characterized by an XRPD pattern substantially in accordance with the pattern of FIG. 2. Crystalline Form A of solifenacin is also characterized by an XRPD pattern having significant peaks at about 3.6, 13.4, 14.1, 15.5, 18.6, 19.2, 20.9, 21.7, and 22.9, ±0.2 degrees 2θ. The pattern is also characterized by additional XRPD peaks at about 17.6 and 18.1, ±0.2 degrees 2θ.

The infrared (IR) spectrum of the crystalline Form A of solifenacin has been recorded on a Perkin Elmer System Spectrum 1 model spectrophotometer, between 450 cm⁻¹ and 4000 cm⁻¹, with a resolution of 4 cm⁻¹ in a potassium bromide pellet, the test compound being at the concentration of 1% by mass.

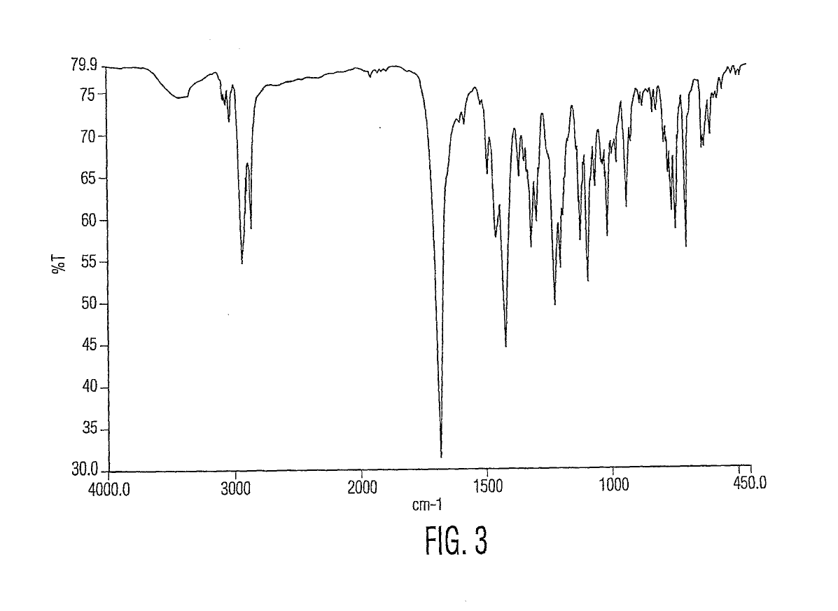

Crystalline Form A of solifenacin is characterized by an infrared absorption spectrum in potassium bromide comprising peaks at about 701, 741, 1014, 1118, 1316, 1462, 1579, 1685, 1950, and 2939, ±5 cm⁻¹. Crystalline Form A of solifenacin is also characterized by its infrared absorption spectrum in potassium bromide substantially in accordance with the spectrum of FIG. 3.

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C.

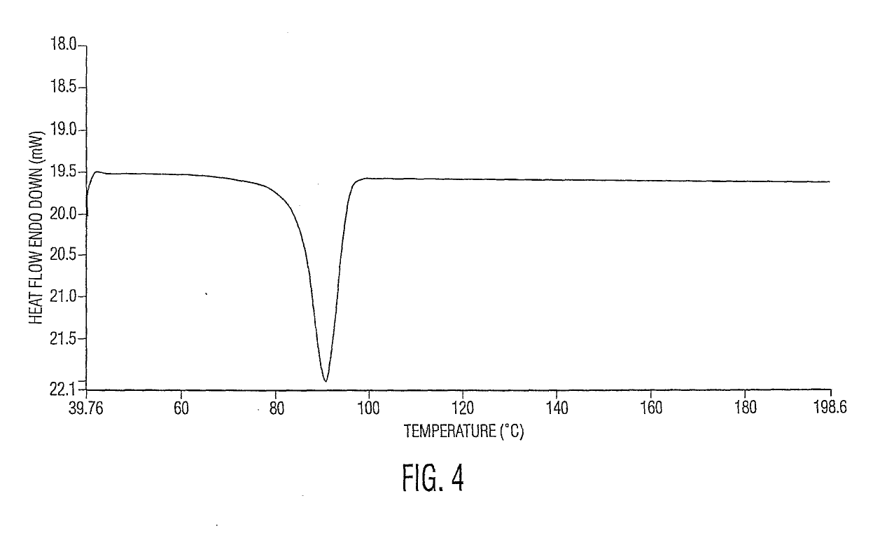

Crystalline Form A of solifenacin has a characteristic differential scanning calorimetry curve substantially in accordance with FIG. 4, having an endotherm at about 90° C. with an onset temperature about 85° C., with Δ H 60 J/g using temperatures from about 40° C. to about 200° C. at the rate of 5° C./minute.

Crystalline Form A of solifenacin prepared according to the present invention has all the characteristics of a normal crystal structure, i.e. it forms a regular crystal lattice. It will be clear to a skilled person that materials which exist in a crystalline state may exist in a number of different polymorphic forms. Polymorphs may be identified by well-known techniques and all polymorphs of solifenacin are envisioned by the present invention.

The crystalline solifenacin has commercially sufficient chemical and polymorphic stability on long-term storage. The crystalline solifenacin can be used in the manufacture of a medicament for the treatment of patients with overactive bladders, urgency and urinary frequency.

Crystalline solifenacin may be combined with a pharmaceutical diluent or carrier to provide pharmaceutical compositions suitable for use in therapy. A formulation comprising crystalline solifenacin has a reduced effective mass of active material compared to a corresponding salt formulation. Therefore, since less material is required to be incorporated into formulations such as tablets, tablet size may be reduced, resulting in improved patient convenience and the likelihood of increased patient compliance.

Another embodiment of the present invention provides a process for the preparation of solifenacin. In a preferred embodiment, the process comprises reacting (1S)-alkyl-1-phenyl-1,2,3,4-tetrahydro-2-isoquinolinecarboxylate of Formula IV with less than about 1.5 molar equivalents of (3R)-3-quinuclidinol of Formula V and less than about 1 molar equivalent of a base, to give solifenacin of Formula VI.

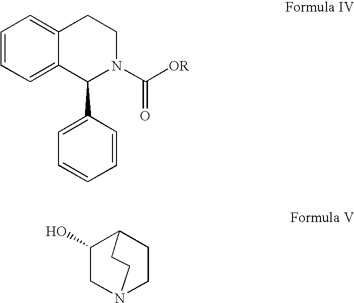

where R is C₁ to C₄ alkyl, aryl, or aralkyl group.

Suitable organic solvents which can be used for the reaction include but are not limited to halogenated solvents such as dichloromethane, ethylene dichloride, chloroform and the like; hydrocarbon solvents such as toluene, xylene, n-heptane, n-hexane, cyclohexane, methylcyclohexane and the like; ethers such as tetrahydrofuran, 1,4-dioxane and the like; aprotic polar solvents such as N,N-dimethylformamide (DMF), dimethylsulfoxide (DMSO), N,N-dimethylacetamide (DMA) and the like; and mixtures thereof.

The mole ratio of (3R)-3-quinuclidinol of Formula V used in the reaction is important in establishing the cost of the process, since it is a very expensive raw material. Only a sufficient amount of raw material should be used so that it is utilized completely in the reaction. The reaction taking place in this step is a condensation reaction, hence an equimolar ratio of the raw materials is required for the reaction to take place. A molar ratio of less than 1.5 moles of (3R)-3-quinuclidinol of Formula IV, per mole of (1S)-alkyl-1-phenyl-1,2,3,4-tetrahydro-2-isoquinolinecarboxylate of Formula IV, is sufficient. Although an excess molar amount of the quinquidinol does not have an impact on the purity of the product, the cost would be increased substantially.

Suitable bases which can be used for the reaction include, but are not limited to alkali metal hydrides such as lithium hydride, sodium hydride and the like. The molar ratio of the base used is important since it determines the percentage of (1S)-1-phenyl-1,2,3,4 tetrahydro-isoquinoline of Formula III remaining as an impurity in the product. Excess base results in breaking of the alkyl chain in (1S)-alkyl-1-phenyl-1,2,3,4-tetrahydro-2-isoquinolinecarboxylate of Formula IV giving back the starting material (1S)-1-phenyl-1,2,3,4 tetrahydro-isoquinoline as an impurity in the product. An optimized ratio of the base which results in the completion of the reaction and does not lead to impurity formation ranges from about 0.5 to 1 mole per mole of (1S)-alkyl-1-phenyl-1,2,3,4-tetrahydro-2-isoquinolinecarboxylate.

The alkali metal hydride which is used as the base in the reaction helps in forming the alkali metal analogue of quinquidinol. A quantity of base which is sufficient to initiate the reaction is enough, since thereafter the alkali metal alkoxide which is formed in-situ in the reaction acts as the base and serves the purpose. Hence the mole ratio of base used need not be 1.0 molar equivalent, even a lower amount is sufficient to initiate the reaction.

Suitable temperatures for conducting the reaction range from about 20° C. to about 200° C., or from about 80° C. to about 150° C. Optionally, after the reaction is complete, the solifenacin formed in the reaction may be isolated, or may be progressed to the next stage without isolation.

In an embodiment of the present invention, the solifenacin formed is isolated in the form of a crystalline solid. Suitable techniques used for isolation include techniques of crystallization, slurrying, or trituration in a suitable solvent.

Suitable solvents which can be used for isolation using the above techniques include, but are not limited to: ketones such as acetone, methyl isobutyl ketone, and cyclohexanone; alcohols such as methanol, ethanol, isopropanol, n-propanol, n-butanol, tertiary-butyl alcohol, ethylene glycol, and the like; chlorinated solvents such as dichloromethane, chloroform, carbon tetrachloride and the like; hydrocarbon solvents such as toluene, xylene, n-hexane, n-heptane, cyclohexane and the like; and mixtures thereof. Depending on the technique chosen, the mixture of solifenacin with the solvent may be in the form of a solution or a suspension. The quantity of solvent used depends on the solvent and the temperature adopted for dissolution if it is a solution. The concentration of solifenacin in the mixture may generally range from about 0.1 to about 1 g/ml in the solvent.

For recrystallization, a solution can be prepared at an elevated temperature if desired to achieve a desired concentration. Any temperature is acceptable for the dissolution as long as a clear solution of the solifenacin is obtained and is not detrimental to the drug substance chemically or physically. The exact temperature required can be readily determined by a person skilled in the art and will also depend on parameters such as concentration. The solution may be brought down to room temperature for further processing if required otherwise; an elevated temperature may be used. A higher temperature will allow the precipitation from solutions with higher concentrations of solifenacin, resulting in better economies of manufacture.

For isolation to occur, the reaction mass may be maintained further at temperatures lower than the concentration temperatures such as for example below about 10° C. to about 25° C., for a period of time as required for a more complete isolation of the product. The exact cooling temperature and time required for complete isolation can be readily determined by a person skilled in the art and will also depend on parameters such as concentration and temperature of the solution or slurry. Optionally, isolation may be enhanced by methods such as cooling, partial removal of the solvent from the mixture, by adding an anti-solvent to the reaction mixture, or a combination thereof.

The solid material isolated is recovered from the final mixture, with or without cooling below the operating temperature, using techniques such as filtration by gravity, or by suction, centrifugation, and the like. The crystals so isolated will carry a small proportion of occluded mother liquor containing a higher percentage of impurities. If desired the crystals can be washed with a solvent to wash out the mother liquor. Optionally, the solid isolated may be further dried. Drying can be carried out at reduced pressures, such as below about 200 mm Hg or below about 50 mm Hg, at temperatures such as about 35° C. to about 70° C. The drying can be carried out for any desired time period that achieves a desired purity, for example, about 1 to 20 hours, or longer. Drying may also be carried out for shorter or longer periods of time depending on the product specifications. The exact time required can be readily determined by a person skilled in the art and will also depend on parameters.

Crystalline solifenacin may also be prepared by treatment of a corresponding salt form of solifenacin under suitable aqueous basic conditions. For example, it may be isolated from the salt by treatment with aqueous base e.g., sodium hydroxide, partitioning the organic and aqueous layers with a suitable organic solvent, separating, drying, and evaporating the organic solution under vacuum.

In another embodiment the present invention provides substantially pure solifenacin.

Solifenacin obtained according to the process of the present invention is substantially pure. By “substantially pure solifenacin” it is meant that solifenacin prepared in accordance with the present invention has a purity of more than about 95%, or more than about 98%, or more than about 99% by HPLC, and contains less than about 2%, or less than about 1%, by weight of the corresponding impurities such as the R,R-isomer of solifenacin, S,S-isomer of solifenacin, (1S)-ethyl-1-phenyl-1,2,3,4-tetrahydro-2-isoquinolinecarboxylate of Formula IV, (1S)-1-phenyl-1,2,3,4-tetrahydro-isoquinoline of Formula II, and all other process related impurities, as characterized by a high performance liquid chromatography (“HPLC”) chromatogram obtained from a mixture comprising the desired compound and one or more of the said impurities.

As used herein “solifenacin R,R-isomeric impurity” refers to (1R)-(3R)-1-azabicyclo[2.2.2]oct-3-yl 3,4-dihydro-1-phenyl-2(1H)-isoquinolinecarboxylate represented by Compound a.

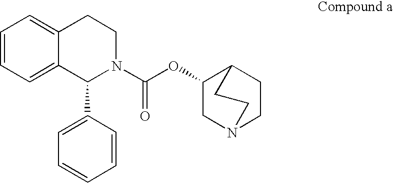

As used herein “solifenacin S,S-isomeric impurity” refers to (1S)-(3S)-1-azabicyclo[2.2.2]oct-3-yl 3,4-dihydro-1-phenyl-2(1H)-isoquinolinecarboxylate represented by Compound b.

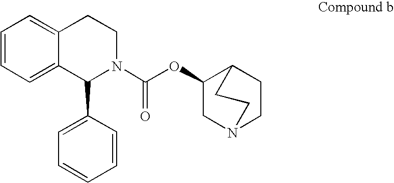

The process of the present invention provides stable crystalline Form A of solifenacin. The term “stable crystalline Form A” refers to stability of the crystalline form under the standard temperature and humidity conditions of testing of pharmaceutical products, wherein the stability is indicated by preservation of the original polymorphic form, and its purity as determined by HPLC.

In another embodiment the present invention provides a process for the preparation of pharmaceutically acceptable salts of solifenacin starting from solid solifenacin, or solifenacin prepared according to the process described above.

Solifenacin obtained by the process of the present invention can be converted to its pharmaceutically acceptable salts by reacting it with the desired acid in the presence of a suitable solvent.

Suitable acids which can be used include, but are not limited to: inorganic acids such as hydrochloric acid hydrobromic acid, and the like; and organic acids such as tartaric acid, succinic acid, acetic acid, citric acid, and the like. Suitable solvents which can be used include, but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane and the like; hydrocarbons such as toluene, xylene, n-heptane, cyclohexane, n-hexane and the like; nitriles such as acetonitrile, propionitrile and the like; and mixtures thereof.

In a preferred embodiment, the acid used is succinic acid, the solvent used is any solvent selected from the classes mentioned above, and the acid addition salt formed is solifenacin succinate.

Solifenacin succinate can also be formed from another acid addition salt of solifenacin, by treatment of the salt under suitable aqueous basic conditions to isolate the free base, which is then treated with succinic acid in the presence of a suitable solvent to form solifenacin succinate.

Solifenacin succinate obtained according to the process of the present invention is characterized by an XRPD pattern substantially in accordance with the pattern of FIG. 2. Crystalline solifenacin succinate obtained is also characterized by an XRPD pattern having significant peaks at about 3.6, 13.4, 14.1, 15.5, 18.6, 21.7, and 22.9, ±0.2 degrees 2θ. The pattern is also characterized by additional XRPD peaks at about 19.2 and 20.9, ±0.2 degrees 2θ.

Solifenacin succinate prepared according to the process of the present invention has chemical and polymorphic stability on long-term storage.

In another embodiment, the present invention provides a process for the preparation of (1S)-alkyl-1-phenyl-1,2,3,4-tetrahydro-2-isoquinolinecarboxylate of Formula IV.

In a preferred embodiment, the process for the preparation of (1S)-alkyl-1-phenyl-1,2,3,4-tetrahydro-2-isoquinolinecarboxylate of Formula IV comprises:

(a) reacting 1-phenyl-1,2,3,4 tetrahydro-isoquinoline of Formula II with an optically pure tartaric acid in the presence of a suitable organic solvent to afford a tartaric acid salt of (1S) 1-phenyl-1,2,3,4 tetrahydro-isoquinoline of Formula IIa;

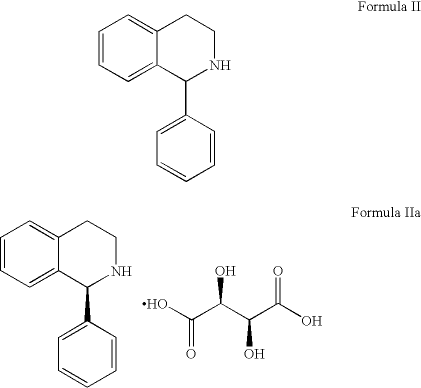

(b) reacting the tartaric acid salt of Formula IIa with a base in presence of a suitable organic solvent to give (1S)-1-phenyl-1,2,3,4 tetrahydro-isoquinoline of Formula III; and

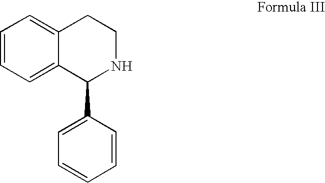

(c) reacting free base of (1S)1-phenyl-1,2,3,4 tetrahydro-isoquinoline of Formula II with alkylchloroformate to give (1S)-alkyl-1-phenyl-1,2,3,4-tetrahydro-2-isoquinolinecarboxylate of Formula IV.

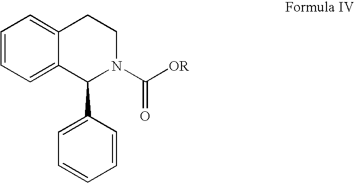

where R is C₁ to C₄ alkyl, aryl, or aralkyl group.

Step (a)—Reacting 1-phenyl-1,2,3,4 tetrahydro-isoquinoline with an optically pure tartaric acid.

The molar ratio of optically pure tartaric acid used for the resolution in step (a) may range from about 1 to about 5 molar equivalents, or from about 1 to about 2 molar equivalents, per molar equivalent of 1-phenyl-1,2,3,4 tetrahydro-isoquinoline.

Suitable solvents which can be used for the resolution include, but are not limited to: alcohols such as ethanol, n-propanol, isopropanol, n-butanol, isobutanol and the like; halogenated solvents such as dichloromethane, ethylene dichloride, chloroform and the like; hydrocarbon solvents such as toluene, xylene, n-heptane, n-hexane, cyclohexane, methylcyclohexane and the like; ethers such as tetrahydrofuran, 1,4-dioxane and the like; aprotic polar solvents such as DMF, DMSO, DMA and the like; and mixtures thereof. Suitable temperatures for conducting the reaction range from about 20° C. to about 100° C., or from about 40° C. to about 80° C.

Step (b)—Reacting the Tartaric Acid Salt of Formula IIa with a Suitable Base.

Suitable bases which can be used in step (b) include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, aqueous solutions containing about 5% to 50%, or about 10% to 20%, (w/v) of the corresponding base can be used. Any concentration which will convert the acid addition salt to a free base may be used.

The pH of the reaction mass may range from about 7 to about 14, or from about 8 to about 10. After the pH is adjusted, the free base can be extracted into an organic solvent. Suitable organic solvents which can be used include, but are not limited to: halogenated solvents such as dichloromethane, ethylene dichloride, chloroform and the like; hydrocarbon solvents such as toluene, xylene, n-heptane, n-hexane, cyclohexane, methylcyclohexane and the like; and ethers such as tetrahydrofuran and the like.

The solid can be isolated from the reaction mass by using techniques similar to that used for isolation of solifenacin.

Step (c)—Reaction of free base of (1S)-1-phenyl-1,2,3,4 tetrahydro-isoquinoline of Formula II with an alkylchloroformate.

Suitable solvents which can be used in step (c) include, but are not limited to: alcohols such as ethanol, n-propanol, isopropanol, n-butanol, isobutanol and the like; halogenated solvents such as dichloromethane, ethylene dichloride, chloroform and the like; hydrocarbon solvents such as toluene, xylene, n-heptane, n-hexane, cyclohexane, methylcyclohexane and the like; ethers such as tetrahydrofuran, 1,4-dioxane and the like; aprotic polar solvents such as DMF, DMSO, DMA and the like; and mixtures thereof. Suitable temperatures for conducting the reaction range from about 10° C. to 100° C., or from about 20° C. to 50° C.

In another embodiment the present invention provides an inexpensive and commercially viable process for the recovery of 1-phenyl-1,2,3,4 tetrahydro-isoquinoline from the mother liquors generated during the resolution stage in the preparation of (1S)-1-phenyl-1,2,3,4-tetrahydro-isoquinoline. In a preferred embodiment, the process comprises treatment of the mother liquor with a suitable base.

The mother liquor is suitably distilled to remove the solvent present in it before proceeding for treatment with the base. The distillation residue may then be dissolved in another suitable solvent, or it may be directly treated with a base. Solvent may be removed by distillation with or without vacuum at elevated temperatures such as about 20° C. to about 70° C. Any temperature and vacuum conditions can be used as long as there is no increase in the impurity levels of the product.

Suitable bases which can be used include, but are not limited to: alkali metal hydrides such as lithium hydride, sodium hydride and the like; alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

Suitably, aqueous solutions containing about 5% to 50%, or about 10% to 20%, (w/v) of the corresponding base can be used. Any concentration which will convert the acid addition salt to a free base may be used.

Suitable solvents which can be used for the reaction include, but are not limited to: alcoholic solvents such as methanol, ethanol, isopropanol, n-butanol, tertiary-butanol, and the like; ethers such as diethyl ether, dimethyl ether, diisopropyl ether, tetrahydrofuran, 1,4 dioxane, and the like; hydrocarbon solvents such as toluene, xylene, and the like; polar aprotic solvents like dimethylformamide, dimethylsulphoxide, dimethylacetamide, and the like; chlorinated solvents like dichloromethane, chloroform, carbon tetrachloride, chlorobenzene and the like; and mixtures of such solvents and water in various proportions.

Suitable temperatures for conducting the reaction range from about 10° C. to about 200° C., or from about 60° C. to about 180° C.

A still further embodiment of the present invention provides a pharmaceutical composition comprising solifenacin or its pharmaceutically acceptable salts prepared according to the process of the present invention, along with one or more pharmaceutically acceptable excipients.

The pharmaceutical composition comprising solifenacin or its salts may be further formulated into solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of the present invention, solifenacin is a useful active ingredient in the range of about 5 to about 50 mg, per dosage unit.

Certain specific aspects and embodiments of this invention are described in further detail by the examples below, which examples are provided only for the purpose of illustration and are not intended to limit the scope of the appended claims in any manner.

### EXAMPLE 1

### Preparation of (1S)-1-phenyl-1,2,3,4-tetrahydro-isoquinoline (Formula III) using methanol as solvent

Methanol (570 ml) and 1-phenyl-1,2,3,4-tetrahydro-isoquinoline of Formula II (95 g) were taken into a clean and dry round bottom flask. D-(−)-tartaric acid (68 g) was added to the above reaction mass and the reaction mass was further heated to about 64° C. The reaction mass was maintained at about 64° C. for about 90 minutes, and then cooled to about 26° C. The reaction mass was then stirred at about 26° C. for about 45 minutes. The separated solid was filtered and washed with methanol (95 ml).

The wet solid was taken into a separate round bottom flask and water (950 ml) and dichloromethane (475 ml) were added, followed by stirring for about 10 minutes. The reaction mass was then cooled to about 26° C. and the pH of the reaction mass was adjusted to about 9 using 8% aqueous sodium bicarbonate solution (190 ml). The resultant reaction suspension was stirred for about 30 minutes, and the organic layer was separated. The aqueous layer was then extracted into dichloromethane (190 ml). The combined dichloromethane layer was washed with water (145 ml) in two lots and the organic layer was then distilled completely at about 56° C. under a vacuum of 300 mm Hg.

Methanol (168 ml) was added to the obtained residue and stirred for about 10 minutes, followed by the addition of D-(−)-tartaric acid (20 g). The reaction mass was heated to about 64° C. and maintained for about 1 hour. The reaction mass was then cooled to about 26° C. and maintained under stirring for about 30 minutes. The separated solid was filtered and washed with methanol (28 ml).

The wet salt was taken into a fresh round bottom flask and water (280 ml) and dichloromethane (140 ml) were added. The reaction mass was stirred at about 26° C. for about 30 minutes and the pH adjusted to about 9.0 with aqueous sodium carbonate solution (7 g in 56 ml). The pH of the reaction solution was adjusted to about 9 using 12% aqueous sodium carbonate solution (70 ml). The reaction mass was stirred at about 26° C. for about 30 minutes, and then the organic layer was separated. The aqueous layer was extracted into dichloromethane (56 ml). The combined organic layer was washed with water (42 ml) in two lots, and the organic layer was distilled completely at about 60° C. under a vacuum of 300 mm Hg. N-Hexane (28 ml) was added to the residue obtained and the mixture was cooled to about 30° C. The mixture was maintained at about 30° C. for about 40 minutes and the separated solid was filtered and washed with n-Hexane (14 ml). The wet solid was dried at about 54° C. for about 2.5 hours to afford 21.5 g of the title compound.

### EXAMPLE 2

### Preparation of (1S)-1-phenyl-1,2,3,4-tetrahydro-isoquinoline (Formula III) using a combination of methanol and ethyl acetate as solvent

1-phenyl-1,2,3,4-tetrahydroisoquinoline (100 g) was taken into a round bottom flask and methanol (400 ml) was added and stirred for about 5 minutes. The reaction mass was then heated to about 40° C., and D-(−)-tartaric acid (71.6 g) was added. The reaction mass was further heated to about 64° C. and maintained for about 2 hours. The reaction mass was then allowed to cool to about 28° C. and ethyl acetate (200 ml) was added. The reaction mass was maintained at about 28° C. for about 20 minutes, and then filtered. The filtered solid was washed with methanol (100 ml) and the wet solid was dried at about 55° C. for about 1 hour, 20 minutes.

The dry material was taken into another round bottom flask and methanol (270 ml) was added. The reaction mass was heated to about 64° C. and maintained for about 1 hour. The reaction mass was then allowed to cool to about 28° C. and ethyl acetate (136 ml) was added. The reaction mass was maintained at about 28° C. for about 1 hour and the solid was filtered and washed with methanol (68 ml). The wet solid was dried at about 50° C. for about 1 hour. The dry solid was taken into another fresh round bottom flask and water (938 ml) was added. The mixture was stirred for about 10 minutes and the pH of the mixture was adjusted to about 8.9 using 10% aqueous sodium hydroxide solution. The mixture was stirred at about 28° C. for about 1 hour and then filtered. The filtered solid was washed with water (125 ml) and dried at about 53° C. for about 9 hours to get 35.9 g of the title compound.

Purity by HPLC: 99.24% by weight.

Chiral purity by HPLC: 99.64% by weight.

### EXAMPLE 3

### Recovery of 1-phenyl-1,2,3,4-tetrahydro-isoquinoline (Formula II) from mother liquors

(1R)-1-phenyl-1,2,3,4-tetrahydro-isoquinoline (5 g) recovered from the filtrate of the resolution step, potassium hydroxide (1.3 g), water (2.5 ml), and dimethyl sulfoxide (50 ml) were taken into a clean and dry round bottom flask and stirred for about 15 minutes. The reaction mixture was heated to about 160° C. and maintained for about 15 hours. Water (50 l) was added to the above reaction mixture and stirred for about 45 minutes. The separated solid was then filtered and washed with water (15 ml). The wet solid was taken into a separate round bottom flask and n-hexane (50 ml) was added and stirred for about 1 hour. The separated solid was filtered and washed with n-hexane (25 ml). The wet solid was dried at about 50° C. to afford 3.2 g of the title compound.

### EXAMPLE 4

### Preparation of 1(S)-phenyl-3,4-dihydro-1H-isoquinoline-2-carboxylic acid ethyl ester (Formula IV) using dichloromethane as solvent

Dichloromethane (180 ml) and (1S)-1-phenyl-1,2,3,4-tetrahydro-isoquinoline (18 g) were charged into a clean and dry round bottom flask. Ethyl chloroformate (13.9 g) was added slowly through a dropping funnel at about 28° C. Water (180 ml) was then added to the reaction mass and the pH of the reaction mass was adjusted to about 2 using 1 N hydrochloric acid (74.3 ml) and stirred for about 10 minutes. The organic layer was separated and the aqueous layer was extracted into dichloromethane (90 ml). The combined organic layer was washed with water (360 ml) in 2 equal lots. The organic layer was then distilled completely at about 55° C. under a vacuum of 300 mm Hg to afford 20.2 g of the title compound as an oil.

### EXAMPLE 5

### Preparation of 1(S)-phenyl-3,4-dihydro-1H-isoquinoline-2-carboxylic acid ethyl ester (Formula IV) using toluene as solvent

(1S)-1-phenyl-1,2,3,4-tetrahydroisoquinoline (50 g) and toluene (500 ml) were taken into a round bottom flask and stirred for about 10 minutes. The reaction mass was then cooled to 0° C. and sodium carbonate (27.8 g) was added. Ethylchloroformate (21.15 ml) was then added at about 2° C. to 3° C. After completing the addition the reaction mass was allowed to reach about 28° C. and maintained for about 2 hours. Reaction completion was determined using thin layer chromatography. After the reaction was complete, the reaction mass was filtered to remove the unwanted solid and the filter bed was washed with toluene (50 ml). The filtrate was washed with water (660 ml) in 2 equal lots. The organic layer was distilled in a Bucchi Rotavapor flask at about 65° C. to give 64.6 g of the title compound.

Purity by HPLC: 98% by weight.

Chiral purity by HPLC: 99.37% by weight.

### EXAMPLE 6

### Preparation of Solifenacin (Formula VI)

Toluene (210 ml) and R-3-quinuclidinol (40.6 g) were taken into a clean and dry round bottom flask and sodium hydride (4.3 g) was added. The reaction mass was stirred for about 10 minutes. 1(S)-Phenyl-3,4-dihydro-1H-isoquinoline-2-carboxylic acid ethyl ester (30 g) dissolved in toluene (90 ml) was added slowly to the above reaction mixture at a temperature of about 26° C. The resultant reaction mixture was heated to about 110° C. and maintained for about 4 hours, 20 minutes. Reaction completion was determined using thin layer chromatography. After the reaction was complete, the reaction mass was cooled to about 26° C. The pH of the reaction mass was adjusted to 2.0 and the organic layer was separated. The aqueous layer was extracted into toluene (90 ml). The pH of the aqueous layer was the adjusted to 7.0 using a 20% aqueous solution of sodium carbonate. Ethyl acetate (300 ml) was added to the aqueous layer and stirred for about 10 minutes. The organic layer was separated and the aqueous layer was extracted into ethyl acetate (150 ml). The combined organic layer was given carbon treatment and distilled off at about 60° C. to get the title compound in the form of an oil.

The compound obtained above (5 g) was taken into a round bottom flask and n-hexane (20 ml) was added. The mixture was stirred at about 26° C. for about 1 hour and 30 minutes. The separated solid was filtered and washed with n-hexane (5 ml). The wet solid was dried at about 50° C. for about 1 hour and 10 minutes to give 3.0 g of the title compound.

Purity by HPLC: 99.0% by weight.
% of the compound of Formula III: 0.33
% of the compound of Formula IV: 0.05
Chiral purity: 97.6% by weight.
% of SS isomeric impurity: 1.7
% of RR isomeric impurity: 0.11

### EXAMPLE 7

### Preparation of Solifenacin (Formula VI)

1(S)-Phenyl-3,4-dihydro-1H-isoquinoline-2-carboxylic acid ethyl ester (100 g) and toluene (500 ml) were taken into a round bottom flask and heated to about 115° C. Moisture was removed from the reaction mass by azeotropic distillation for about 3 hours. Then the reaction mass was cooled to about 55° C. and (3R)-3-quinuclidinol (54.23 g) was added. Again the reaction mass was heated to about 115° C. and maintained for about 2 hours. The reaction mass was cooled to about 55° C. and sodium hydride (2.81 g) was added. Again the reaction mass was heated to about 115° C. and maintained for about 4 hours. Solvent (50 ml) was removed from the reaction mass by distillation and fresh toluene (50 ml) was added. The reaction mass was maintained at about 115° C. for about 3 hours, and again solvent (50 ml) was removed from the reaction mass and fresh toluene (50 ml) was added. The reaction mass was maintained at about 115° C. for about 3 hours and again solvent (50 ml) was removed from the reaction mass and fresh toluene (50 ml) was added. The reaction mass was then cooled to about 28° C. and saturated aqueous sodium chloride solution (200 ml) was added. The organic layer was separated and washed with water (400 ml). The organic layer was then extracted into a 20% aqueous hydrochloric acid solution (1000 ml). The aqueous layer was then washed with toluene (100 ml). The aqueous layer was cooled to about 15° C. and the pH was adjusted to 10 using an aqueous 20% sodium hydroxide solution (500 ml). Ethyl acetate (500 ml) was added to the aqueous layer and stirred for about 10 minutes. The organic layer was separated and the aqueous layer was extracted into ethyl acetate (500 ml). The combined organic layer was washed with water (200 ml) in two equal lots. The organic layer was distilled at about 55° C. to give 115 g of the title compound.

Purity by HPLC: 90.71% by weight.

Chiral purity: 93.3% by weight.

### EXAMPLE 8

### Preparation of Solifenacin Succinate from Solid Solifenacin (Formula I)

Ethanol (10 ml), ethyl acetate (7.5 ml) and solifenacin (5 g) were taken into a clean and dry round bottom flask and stirred for about 15 minutes. Succinic acid (1.8 g) was added and stirred for about 10 minutes. The reaction mass was then heated to about 75° C. and maintained for about 2.5 hours. The reaction mass was then gradually cooled to about 4° C. and maintained for about 4 hours. The separated solid was filtered and washed with ethanol (5 ml). The wet solid was dried at about 53° C. for about 3 hours to afford 3.8 g of the title compound.

Purity by HPLC: 99.1% by weight.

### EXAMPLE 9

### Preparation of Solifenacin Succinate (Formula VII)

Solifenacin (25 g) and acetone (200 ml) were taken into a round bottom flask and stirred for about 15 minutes at about 28° C. The reaction mass was filtered and the filtrate was taken into a separate round bottom flask. Succinic acid (8.149 g) was added to the above filtrate under stirring. The reaction mass was then heated to about 60° C. and maintained for about 1 hour. The reaction mass was then cooled to about 12° C. and maintained for about 1 hour. The separated solid was filtered and the filtered solid was washed with about 25 ml of acetone. The wet solid was taken into another round bottom flask and heated to about 60° C. The reaction mass was maintained at about 60° C. for about 1 hour and then cooled to about 10° C. The reaction mass was maintained at about 10° C. for about 1 hour. The separated solid was filtered and washed with acetone (25 ml). The wet solid was dried at about 50° C. for about 5 hours to yield 25.7 g of the title compound.

Purity by HPLC: 99.78% by weight.

### EXAMPLE 10

### Preparation of Solifenacin Hydrochoride (Formula VII)

Solifenacin (15 g) was taken into a round bottom flask and ethanol (164.5 ml) was added and stirred. A solution of 4N solution of hydrochloric acid (11.75 ml) in ethyl acetate was added to the above reaction mass. The obtained reaction mass was transferred into a Buchi Rotavapor and distilled to dryness at about 60° C. The obtained residue was cooled to about 30° C., and acetonitrile (62 ml) was added and stirred at about 30° C. for about 5 minutes. Diethyl ether (166.3 ml) was then added, and stirred for about 5 hours. The separated solid was filtered and washed with about 10 ml of diethyl ether. The wet solid was taken into another round bottom flask and acetonitrile (150 ml) was added and stirred for about 10 minutes. The reaction mass was then heated to about 60° C. followed by cooling to about 40° C., then diethyl ether (234 ml) was added at about 25° C. The reaction mass was stirred at a temperature of about 25° C. for about 5 hours. The separated solid was then filtered and washed with diethyl ether (15 ml) and suction dried under a nitrogen atmosphere to afford 5 g of the title compound.

Purity by HPLC: 99.13% by weight.

### EXAMPLE 11

### Preparation of Solifenacin Succinate from Solifenacin Hydrochloride (Formula I)

Solifenacin hydrochloride (2 g) was taken into a round bottom flask and water (10 ml) and dichloromethane (30 ml) were added. The mixture was stirred and 5% aqueous sodium bicarbonate solution was added. The organic layer was separated and washed with water (10 ml). The organic layer was then distilled at a temperature of about 40° C. and the residue was cooled to about 30° C. Methanol (20 ml) was added and the reaction mass was stirred for about 10 minutes followed by addition of succinic acid (0.52 g). The reaction mixture was then subjected to distillation at a temperature of about 60° C. and the obtained residue was cooled to about 30° C. Acetonitrile (6.62 ml) and diethyl ether (17.7 ml) were added to the residue and subjected to stirring until the solid separated. The separated solid was filtered and washed with diethyl ether (2 ml) and subjected to suction drying at about 28° C. to afford 1.5 g of the title compound.

Purity by HPLC: 99.74% by weight.

### EXAMPLE 12

### Preparation of Solifenacin Succinate from Crude Solifenacin Using Acetone as Solvent

Solifenacin succinate (20 g), succinic acid (6.5 g), and acetone (400 ml) were taken into a round bottom flask and stirred at about 28° C. The reaction mass was heated to about 56° C. and maintained for about 30 minutes. Acetone (200 ml) was again added to the reaction mass in two equal lots at about 56° C. to obtain a clear dissolution. Once a clear solution was obtained, the reaction mass was cooled to about 28° C. and filtered. The filtrate was divided into two equal parts.

Part I: The solvent was distilled at about 48° C. for about 20 minutes (about 15% of the solvent was distilled). The remaining residue was allowed to cool to about 28° C. and maintained for about 1 hour. The separated solid was filtered and the wet solid was dried at about 50° C. for about 6 hours to yield 6.2 g of the title compound.

Purity by HPLC: 99.32% by weight.

Part II: The filtrate was allowed to cool to about 28° C. and maintained for about 1 hour. The separated solid was filtered and the wet solid was dried at about 50° C. for about 6 hours to yield 5.8 g of the title compound.

Purity By HPLC: 99.48% by weight.

### EXAMPLE 13

### Preparation of Solifenacin Succinate from Crystalline Form A of Solifenacin

Crystalline Form A of solifenacin (5 g) was taken into a round bottom flask and ethanol (51.7 ml) was added and stirred for about 5 minutes. Ethyl acetate (3.69 ml) and succinic acid (1.629 g) were added to the above reaction mass and stirred at about 30° C. for about 2 hours. The reaction mass was then taken into a Buchi Rotavapor flask and distilled at about 60° C. Acetonitrile (20.7 ml) was added to the residue and stirred for about 10 minutes. Diethyl ether (55.5 ml) was then added to the above reaction mass and stirred for about 2 hours. The separated solid was filtered and washed with diethyl ether (5 ml). The wet solid was then taken into another round bottom flask and acetonitrile (20.7 ml) was added. The reaction mass was heated to about 60° C. and then cooled to about 38° C. and diethyl ether (78 ml) was added. The reaction mass was further cooled to about 25° C. and maintained for about 2 hours. The separated solid was filtered and washed with diethyl ether (5 ml). The wet solid was dried at about 50° C. for about 4 hours to yield 4.3 g of the title compound.

### EXAMPLE 14

### Stability of Crystalline Form A of Solifenacin

Solifenacin prepared according to the process given in Example 9 was packaged in a self-sealing polyethylene bag. The material was stored for 3 months at room temperature under normal atmospheric conditions and checked for polymorphic stability. The material was found to retain its polymorphic form after three months of holding, as indicated by maintenance of the original XRPD pattern and original purity.

1. A process for preparing solifenacin having Formula VI, 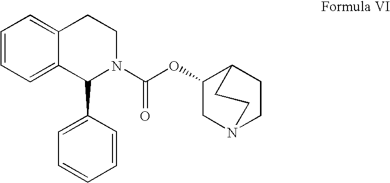
or a salt thereof, which process comprises:
reacting the compound having Formula IV 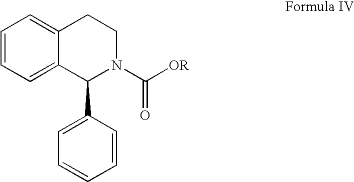
where R is a C₁ to C₄ alkyl, aryl, or aralkyl group, with less than about 1.5 molar equivalents, per mole of Formula IV, of a compound having Formula V 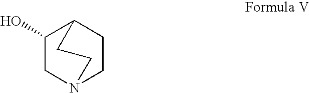
in the presence of less than about 1 molar equivalents of a base per molar equivalent of the compound having Formula IV.

2. The process of claim 1, wherein the base used is an alkali metal hydride.

3. The process according to claim 2, wherein the base used is sodium hydride.

4. The process according to claim 1, further comprising recovering solifenacin in the form of a crystalline solid.

5. An isolated solid form of Solifenacin.

6. The solid solifenacin of claim 5, having an XRPD pattern substantially in accordance with the pattern of FIG. 2.

7. Solifenacin of claim 5 having a purity of more than about 95%.

8. Solifenacin of claim 5 having a purity of more than about 99%.

9. Solifenacin, containing less than about 2 area-% by high performance liquid chromatography of each of the impurities having the Formulae a and b:

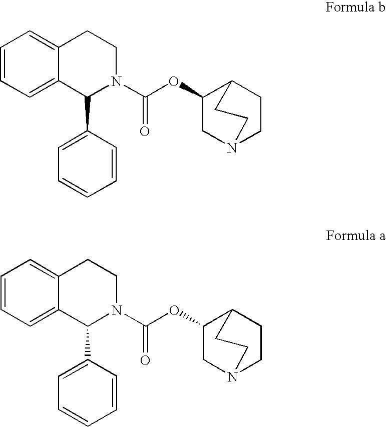

10. A process for preparing solifenacin or a salt thereof, which process comprises:
(a) reacting a compound having a Formula II 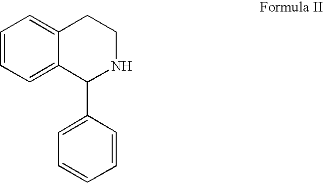
with an optically pure tartaric acid, to give a solid tartaric acid salt having a Formula IIa; 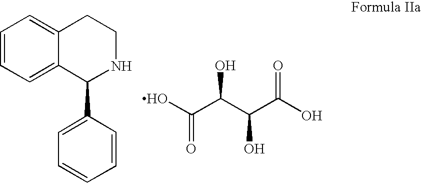
(b) reacting said tartaric acid salt with a base, to form a compound having a Formula III; 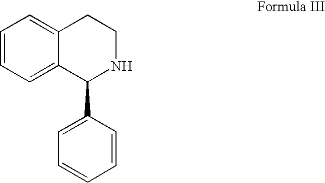
(c) reacting a compound of Formula III with an alkylchloroformate to give the compound of Formula IV, 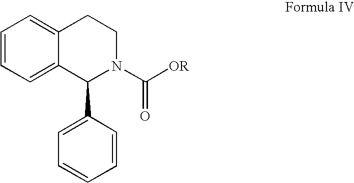
where R is an alkyl group; and
(d) reacting a compound of Formula IV with a compound of Formula V, 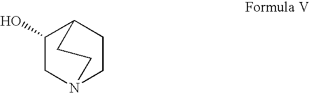
to form solifenacin.

11. The process of claim 10, wherein said alkylchloroformate is ethylchloroformate.

12. The process of claim 10, wherein the compound of Formula III is isolated as a solid.

13. The process of claim 10, further comprising the step of treating a mother liquor from isolating a solid compound of Formula IIa with a base to form a compound of Formula II.

14. A process for preparing a pharmaceutically acceptable salt of solifenacin, which comprises reacting the compound obtained by the process of claim 1 with a pharmaceutically acceptable acid in the presence of an organic solvent.

15. A process for preparing a pharmaceutically acceptable salt of solifenacin, comprising reacting the compound obtained by the process of claim 10 with a pharmaceutically acceptable acid in the presence of an organic solvent.

16. The process of claim 14, wherein said pharmaceutically acceptable acid addition salt is succinic acid.

17. The process of claim 15, wherein said pharmaceutically acceptable acid addition salt is succinic acid.

18. The process of claim 14, wherein the organic solvent is a ketone.

19. The process of claim 15, wherein the organic solvent is a ketone.

20. The process of claim 18, wherein said ketonic solvent is acetone.

21. The process of claim 19, wherein said ketonic solvent is acetone.

22. (canceled)

23. The process of claim 10, further comprising recovering solifenacin as a solid.
